(19) United States
(12) Patent Application Publication
(10) Pub. No.: US 2005/0089954 A1
(43) Pub. Date: Apr. 28, 2005

(54) Gene cluster for rabelomycin biosynthesis and its use to generate compounds for drug screening

(76) Inventors: Kaisa Palmu, Turku (FI); Tero Kunnari, Forby (FI)

Correspondence Address: BIRCH STEWART KOLASCH & BIRCH, PO BOX 747, FALLS CHURCH, VA, 22040-0747, US

(21) Appl. No.: 10/469,442

(22) PCT Filed: Mar. 15, 2002
(86) PCT No.: PCT/FI02/00214

(30) Foreign Application Priority Data
Mar. 19, 2001 (FI) 20010553

(51) Int. Cl. C12P 21/02; C07H 21/04; C12P 19/30; C12N 1/21

(52) U.S. Cl. 435/69.1; 435/89; 435/320.1; 435/252.3; 536/23.2; 435/471

(57) ABSTRACT

This invention relates to the gene cluster for angucycline biosynthesis, derived from Streptomyces, and use of the genes therein to obtain antibiotics for drug screening.

This invention relates to the gene cluster for angucycline biosynthesis, derived from Streptomyces, and use of the genes therein to obtain antibiotics for drug screening.

### BACKGROUND OF THE INVENTION

Tetracyclic aromatic polyketides known as angucyclines were first isolated from bacterial cultures over thirty years ago. The angucycline group of antibiotics has become a rapidly growing group of bioactive natural products, whose members are discovered by diverse screening methods such as antibacterial, antitumor and chemical screens.

These compounds are biosynthetized in microbes by polyketide pathway by type II polyketide synthase. The polyketide is folded in a manner characteristic to angucyclines: the fourth ring is orientated in an angular fashion, as described by the name ‘angucycline’. The aglycone formed is subsequently modified by diverse reactions, such as oxidation, hydroxylation and glycosylation at various positions, to give a variety of structures. Furthermore, chemical synthesis to create angucyclines for drug discovery purposes has been described. Biosynthesis gene clusters for a few angucycline antibiotics have been cloned and partially characterized; the clusters for urdamycin from Streptomyces fradiae, landomycin from S. cyanogenus S136, jadomycin from S. venezuelae ISP5230, and pradimicin from Actinomadura verrucosospora (Decker et al., 1995, Westrich et al., 1999, Han et al., 1994, Dairi et al., 1999, respectively). The clusters for kinamycin from S. murayamaensis (Gould et al., 1998), tetrangulol and tetrangomycin from S. rimosus and PD 116740 from the Streptomyces strain WP 4669 (Hong et al., 1997) have been cloned and expressed in heterologous hosts. The gene cluster for pradimicin is disclosed in an international patent application of Oki et al. (WO 98/11230).

The angucycline antibiotics exhibit diverse bioactivities. Besides an antitumor activity, some of the angucyclines act as enzyme inhibitors, potent inhibitors of blood platelet aggregation, and most of them exhibit antimicrobial activity. In vivo cytostatic activities were reported for the kerriamycins and antibiotic SS-228Y, which can prolong the survival periods of mice inoculated with Erlich ascites tumors. Vineomycins exhibit antitumor activity against Sarcoma 180 solid tumor in mice. Remarkably, some of the members of the angucycline group have been described as inhibiting the growth of cell lines resistant to various cytostatics in market.

For the literature of the angucycline group concerning chemical synthesis, biosynthesis, bioactivites and the molecular structures, see the reviews by Krohn and Rohr (1997) and by Rohr and Thiericke (1992) and the references therein.

### SUMMARY OF THE INVENTION

The present invention concerns a gene cluster derived from Streptomyces bacteria, especially that of the strain Streptomyces sp. H021, which is involved in angucycline biosynthesis. The strain used for gene cloning failed to produce angucyclines in several culture conditions tested in our laboratory. However, expressing a DNA fragment of said cluster in S. lividans or in S. coelicolor, rabelomycin, 5-OH-rabelomycin, and a novel compound, 11-OH-rabelomycin, were obtained. These compounds are members of the angucycline group. Furthermore, when the cluster was introduced into the Streptomyces hosts S. argillaceus and S. galilaeus, they generated a novel compound, 9-O-methyl-rabelomycin, and a prior known compound, ε-rhodomycinone, respectively.

Consequently, a primary object of the invention is the DNA fragment which is the gene cluster for rabelomycin biosynthetic pathway of Streptomyces bacteria, which fragment is included in two 9.5 kb flanked PstI fragments of Streptomyces genome. Further objects of the invention are a recombinant DNA comprising said DNA fragment, and a process for production of hybrid compounds, specifically hybrid anthracyclines and aromatic polyketides, by transferring the DNA fragment of the invention into a Streptomyces host to obtain angucyclines for drug screening.

### DETAILED DESCRIPTION OF THE INVENTION

The experimental procedures used in the present invention are methods conventional in the art. The techniques not explained in detail here are given in the manuals by Hopwood el al. “Genetic manipulation of Streptomyces: a laboratory manual”, The John Innes Foundation, Norwich (1985) and by Sambrook et al. (1989) “Molecular cloning: a laboratory manual”. The publications, patents and patent applications cited herein are given in the reference list in their entirety, and they are incorporated herein by reference.

The present invention concerns particularly the gene cluster for the angucycline biosynthesis (11P2), causing the production of rabelomycin and its derivatives in S. lividans, a non-producer of angucyclines. In specific, the invention concerns the use of the genes for rabelomycin biosynthesis to generate hybrid products modified in several positions when expressed in S. lividans, or in S. argillaceus, a producer of mithramycin. Furthermore, the invention concerns the gene fragment 11P23, that contains genes involved in sugar biosynthesis.

The biosynthetic genes for angucyclines can be isolated from Streptomyces spp., particularly from such strains which give a positive hybridization signal by a short fragment of ketosynthase I (KS I) for rabelomycin biosynthesis. Since these genes were silent in the donor strain Streptomyces sp. H021 used in our experiments, it will be appreciated that as a donor any actinomycete, especially a streptomycete bacterium can be used, obtained by screening with DNA-fingerprinting techniques with the primers similar to rabelomycin KS I gene.

A bacterial strain carrying the genes for rabelomycin can be isolated from a soil sample by any conventional screening method, but especially DNA fingerprinting of polyketide (Type II) is suitable. The primers for DNA fingerprinting are degenerated nucleotide oligomers sharing the sequences 5′-TSGCSTGCTTCGAYGSATC-3′ (SEQ ID NO:21) and 5′-TGGAANCCGCCGAABCCGCT-3′ (SEQ ID NO:22). The bacterial strain that gave a DNA fragment similar to angucyclines in PCR reaction, using the primers as described, was used to deliver DNA for the construction of the gene library.

Genomic DNA of a Streptomyces strain containing the genes for rabelomycin biosynthesis is used in preparing a gene library. Suitable gene fragments for cloning may be obtained by any frequently digesting restriction enzyme. Typically Sau3AI is used. The isolated fragments can be inserted by ligation in any Escherichia coli vector, such as a plasmid, a phagemid, a phage, or a cosmid, though a cosmid vector is preferred, since it enables cloning of large DNA fragments. A cosmid vector, such as pFD666 (ATCC Number 77286) is suitable for this purpose, as it enables cloning of fragments of about 40 kb. BamHI site of pFD666, giving sticky ends to the Sau3AI fragments, may be used for cloning. To package a ligation mixture containing recombinant cosmids in phage particles, commercially available kits may be used. Several E. coli strains can be used for infection by the recombinant cosmids packaged, and a suitable one is e.g. E. coli XL1 Blue MRF′, deficient in several restriction systems.

Using E. coli as a host strain for a gene library, hybridization is an advantageous screening strategy. The probe for hybridization may be any known fragment derived from the rabelomycin gene cluster but a short fragment of 613 nt, prepared by multiplying a region from ketosynthase I with degenerated primers, is preferred. Colonies for the gene library are transferred to membranes for filter hybridization, and nylon membranes are typically used. Any method for detection for hybridization may be used but, in particular, the DIG System (Boehringer Mannheim, GmbH, Germany) is useful. Since the probe is homologous to the hybridized DNA, it is preferable to carry out stringent washes of hybridization at 68° C. in a low salt concentration, according to Boehringer Mannheim's manual, DIG System User's Guide for Filter Hybridization. At least 80%, preferably 90%, homology is suggested to be needed for a DNA fragment to be bound to a probe in the conditions used for washes.

Using this protocol, two clones out of about 1000 gave positive signals and were picked up for DNA isolation. Restriction mapping is an appropriate technique for characterizing the clones. The positive clones may be digested with convenient restriction enzymes to demonstrate the physical linkage map of the DNA fragments. We designated the positive clones obtained as pFDH0211.1 and pFDH0216.1. In expression studies we preferred to use pIJ486, a high copy number Streptomyces plasmid. However, any plasmid which is able to stably replicate in Streptomyces may be used. The clone pFDH0211.1 was transferred into S. lividans TK24 as two PstI-fragments inserted into pIJ486. The two recombinant plasmids obtained were designated as pS11P2 and pS11P23 containing 9.5 kb fragments from H021 genomic DNA. These were further introduced into other Streptomyces strains by protoplast transformation.

In TK24 the plasmid pS11P2 caused the production of rabelomycin and its 5-OH and 11-OH derivatives. A further introduction into S. argillaceus caused the production of 9-O-methylrabelomycin. In addition, when expressed in S. galilaeus H039, which produces aklavinone-rhodinose-rhodinose, the plasmid generates the production of 11-OH-akla-vinone, also called ε-rhodomycinone, with corresponding sugars, suggesting that 11-hydroxylation activity is caused by a gene included in pS11P2. The plasmid pS11P23 caused the production of typical aclacinomycins in S. galilaeus H075, which endogenously produces aklavinone-rhodosamine-deoxyfucose-deoxyfucose. The variety of the modifications in the Streptomyces strains used as hosts give promising usefulness of the genes for combinatorial biosynthesis, to create novel compounds and new chemical structures for drug discovery.

The sequence analysis can be made by any computer-based program, such as GCG (Madison, Wis., USA) package. Sequencing of the two flanking fragments, 11P2 and 11P23, used for cloning, consisting of 19016 bp, revealed 17 complete ORFs.

According to the present invention the putative gene functions as deduced from the sequence homologies of those available in gene banks are: the orfs A, B and C code for minimal polyketide synthase (minPKS), ketosynthase I and II (KSI and KSII) and acyl carrier protein (ACP), respectively; orfD codes for polyketide ketoreductase; orfs E and M code for oxygenases; orfs F and L code for polyketide cyclases; orfs V and O code for reductases; orfH codes for dTDP-glucose-4,6-dehydratase; orfQ codes for NDP-hexose-3-dehydroxylase; orfs (partial) codes for NDP-hexose-2,3-dehydratase; orfR codes for 4-ketohexose reductase; orfR1 (partial) codes for a regulatory gene; orfJ codes for a transporter involved in resistance; orfl codes for protein of unknown function and orf2 codes for an oxidoreductase (see Table 1).

Streptomyces strains, in particular S. lividans, S. argillaceus and S. galilaeus, carrying the recombinant plasmids, are cultivated in media which enable antibiotic production. The compounds, rabelomycin and its derivatives, aclacinomycin and ε-rhodomycinone, are extracted with organic solvents from the culture broth, and the compounds are separated and purified using chromatographic techniques.

According to this invention the strain S. lividans TK24 carrying the plasmid pS11P2, and designated as TK24/pS11P2, produces rabelomycin, 5-OH-rabelomycin and 11-hydroxy-rabelomycin in El medium, supplemented with thiostrepton to give selection pressure for the plasmid containing strains. The strain S. lividans TK24/pS11P2 and the strain TK24/pS11P23, carrying the plasmid containing the flanking region to 11P2, were deposited according to the Budapest Treaty at Deutsche Sammlung von Mikroorganismen und Zelikulturen GmbH (DSMZ), Mascheroder Weg 1b, D-38124 Braunschweig, Germany on 13 Mar. 2001 with the accession numbers DSM 14172 and DSM 14173, respectively.

Any DNA fragment of the invention subcloned from a 19 kb rabelomycin biosynthesis region can be inserted into a vector replicating in Streptomyces, and the products may be obtained by fermentation of the strains carrying the plasmids.

### BRIEF DESCRIPTION OF THE DRAWINGS

FIG. 1. shows the structures of rabelomycin (1), 9-O-methyl-rabelomycin (2), 5-OH-rabelomycin (3), 11-OH-rabelomycin (4), 19-methyl-SEK15 (5) and ε-rhodomycinone (6). The ring numbering used is also given.

FIG. 2 shows the gene cluster (11P232) of the invention. The PstI fragment from 1 to 9652 is the fragment 11P23 for complementation of the mutant H075, and the fragment from 9647 to 19016 is the fragment 11P2 for rabelomycin biosynthesis.

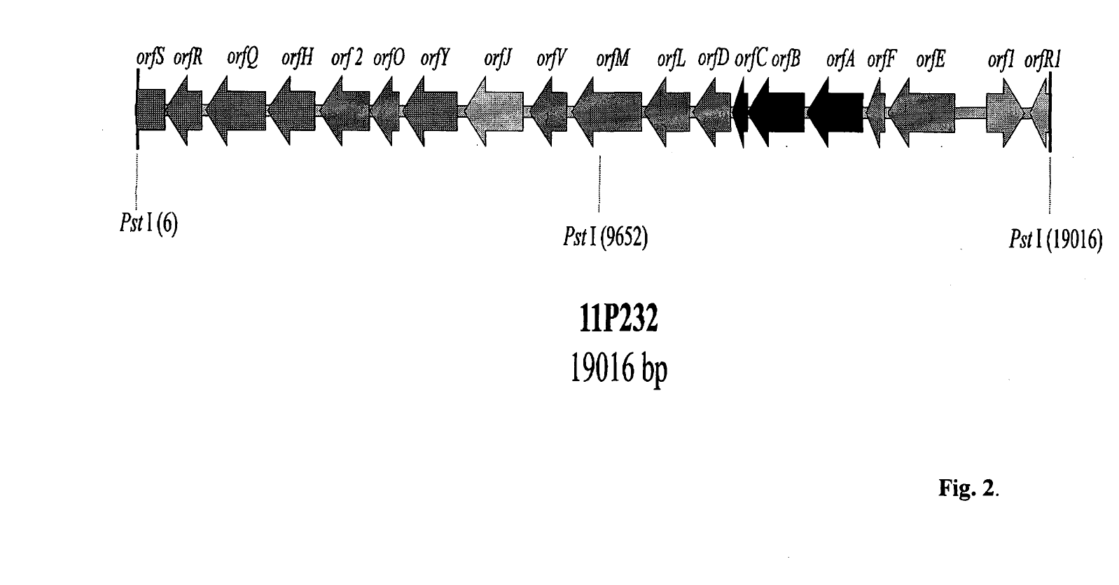

Examples to further illustrate the invention are given hereafter.

### EXPERIMENTAL

Materials Used

Restriction enzymes used were purchased from Promega (Madison, Wis., USA) or Boehringer Mannheim (Germany), alkaline phosphatase from Boehringer Mannheim, and used according to the manufacturers' instructions. Proteinase K was purchased from Promega (Madison, Wis., USA) and lysozyme from Sigma (St. Louis, Mich., USA). Hybond™-N nylon membranes used in hybridization were purchased from Amersham (Buckinghamshire, England), DIG DNA Labelling Kit and DIG Luminescent Detection Kit from Boehringer Mannheim. Qiaquick Gel Extraction Kit from Qiagen (Hilden, Germany) was used for isolating DNA from agarose. Templates for sequencing were prepared using Template Generation System F-700 (Finnzymes, Finland) and the DNA sequencing was performed using the automatic ABI DNA sequenator (Perkin-Elmer) according to the manufacturer's instructions.

Bacterial Strains and Their Use

Escherichia coli XL1 Blue MRF′ (Stratagene, La Jolla, Calif.) was used for cloning. Streptomyces sp. H021 was isolated from a soil sample collected from Turku, Finland and was studied due to the polyketide DNA-fingerprints obtained by the course of our genetical based screening for polyketide producers. The gene cluster of rabelomycin biosynthesis was cloned from this strain.

The host strains to express the genes cloned were:
Streptomyces lividans TK24 (U.S. Pat. No. 5,986,077). This strain was also used as a primary host to clone DNA propagated in E. coli.
Streptomyces galilaeus H075, DSM 11638, (FI 105554 B) produces aklavinone-rhodos-amine-2-deoxyfucose-2-deoxyfucose.
Streptomyces galilaeus H039 (Ylihonko et al. 1994) produces aklavinone-rhodinose-rhodinose-rhodinose.
Streptomyces argillaceus ATCC 12956 produces mithramycin.
   Plasmids

E. coli—Streptomyces shuttle cosmid pFD666 (ATCC 77286) was used for cloning the chromosomal DNA. E. coli cloning vector and pUC19 was used for making the sub-clones.

pIJ486 is a high copy plasmid vector provided by prof Sir David Hopwood, John Innes Centre, UK (Ward et al., 1986). To clone the probe TOPO TA Cloning Kit (Invitrogen, USA) was used according to the manufacturer's instructions.

Nutrient Media and Solutions

For cultivation of the strain H021 for total DNA isolation TSB medium was used. Lysozyme solution (0.3 M sucrose, 25 mM Tris, pH 8, and 25mM EDTA, pH 8) was used in the isolation of total DNA. TE buffer (10 mM Tris, pH 8.0 and 1 mM EDTA) was used to dissolve DNA.

Tryptone Soya Broth (TSB)

Per litre: Oxoid Tryptone Soya Broth powder 30 g.

ISP4

Bacto ISP-medium 4, Difco; 37 g/l.

E1

| | | |
|---|---|---|
| Per liter in tap water: | glucose | 20 g |
| | soluble starch | 20 g |
| | Farmamedia | 5 g |
| | Yeast extract | 2.5 g |
| | K₂HPO₄.3H₂O | 1.3 g |
| | MgSO₄.7H₂O | 1 g |
| | NaCl | 3 g |
| | CaCO₃ | 3 g |

pH adjusted to 7.4 before autoclaving

General Methods

Polyketide metabolites were detected by TLC (Kieselgel 60 F₂₅₄ glass plates) and HPLC on a Hewlett Packard instrument (1100 series) using a Zorbax column (SB-C1 8, 3 μm, 4.6×150 mm) and gradient elution with a mixture of MeCN—H₂O—HCO₂H (30:70:1).

NMR spectra were acquired on a JEOL JNM-GX 400 spectrometer equipped with either a 5 mm normal configuration CH probe or a 5 mm inverse HX probe operating at 400 MHz for ¹H and 100 MHz for ¹³C. The spectra were run at 26° C. in the solvents indicated in Tables 2 to 4, and both ¹³C and ¹H were referenced internally to TMS, assigned as 0 ppm. Electron impact mass spectrometry spectra were taken on a VG Analytical Organic mass spectrometer 7070 E.

ISP4 plates supplemented with thiostrepton (50 μg/ml) were used to maintain the plasmid carrying cultures.

### EXAMPLE 1

### Cloning the Gene Cluster for Rabelomycin Biosynthesis

1.1 Cosmid Library

For isolation of total DNA, the strain H021 was grown for three days in 50 ml of TSB medium supplemented with 0.5% glycine. The cells were harvested by centrifuging for 15 min at 3900×g in 12 ml Falcon tubes, and the cells were stored at −20° C. Cells from a 12 ml sample of the culture were used to isolate the DNA. 5 ml of lysozyme solution containing 5 mg/ml lysozyme was added onto the cells, and incubated for 20 min at 37° C. 500 μl of 10% SDS containing 1 mg of proteinase K was added onto the cells, and incubated for 90 min at 62° C. The sample was chilled on ice and 600 μl of 3M NaAc, pH 5.8 was added, and the mixture was extracted with equilibrated phenol (Sigma). The phases were separated by centrifuging at 1400×g for 10 min. The DNA was precipitated from the water phase with an equal volume of isopropanol, collected by spooling with a glass rod and washed by dipping into 70% ethanol, air dried and dissolved in 500 μl of TE-buffer.

The chromosomal DNA was partially digested with Sau3AI. The DNA fragments were separated by agarose gel electrophoresis and the fragments of 30 to 50 kb were cut from the 0.3% low gelling temperature SeaPlaque® agarose. The DNA bands were isolated from the gel by heating to 65° C., extracting with an equal volume of equilibrated phenol and the phases were separated by centrifuging for 15 min at 2500×g. The phenol phase was extracted with TE buffer, centrifuged and the water phases were pooled. The DNA was precipitated by adding 0.1 volume of NaAc, pH 5.8 and 2 volumes of ethanol at −20° C. for 30 min, centrifuged for 30 min at 15 000 rpm in Sorvall RC5C centrifuge, using SS-34 rotor with adapters for 10 ml tubes. The pellet was air dried and dissolved in 20 μl of TE buffer. The isolated fragments were ligated to pFD666 cosmid vector digested with BamHI and dephosphorylated. The DNA was packed into phage particles and infected to E. coli using Gigapack® III XL Packing Extract Kit according to the manufacturer's instructions.

1.2 Identification of the Clones by Hybridization

The infected cells were grown on LB plates containing 50 μg/ml kanamycin and transferred to Hybond™-N nylon membranes (Amersham). DNA was attached to membranes according to the protocol described in Boehringer Mannheims manual “The DIG System User's Guide for Filter Hybridization”. The probe used to screen the colonies for the biosynthesis cluster was prepared by multiplying a part of the ketosynthase gene with degenerated primers as described by Metsä-Ketelä et al. (1999), and cloned using TOPO TA Cloning Kit (Invitrogen, USA). The plasmid carrying the probe was digested with EcoRI and the fragment was separated from the vector by agarose gel electrophoresis and isolated from the gel using Qiaquick Gel Extraction Kit (Qiagen). The probe was labelled by digoxygenin according to Boehringer Mannheim's manual “The DIG System User's Guide for Filter Hybridization”. Approximately 1000 colonies were screened by hybridization at 68° C., using the probe described. Positive colonies were detected using DIG Luminescent Detection Kit (Boehringer Mannheim). Two colonies gave a positive signal. These clones were designated as pFDH0211.1 and pFDH0216.1. Cosmids from the positive clones were isolated from a 5 ml culture by alkaline lysis method. Restriction analysis showed that the cloned fragmens overlapped each other, representing at least 50 kb of the continuous DNA.

1.3 Subcloning the Fragments for Sequencing

The clone pFDH0211.1 was digested with PstI, and two fragments of about 9.5 kb were isolated and ligated to pUC19 that had been digested with PstI and dephosphorylated. These two fragments are located next to each other in the H021 genome. The clones were named as p11P2 and p11P23, and they were used as templates for sequencing, using Template Generation System F-700 (Finnzymes, Finland). A subclone partially overlapping the fragments 11P2 and 11P23 that was prepared from pFDH0211.1 was also sequenced, and this sequence confirmed that the fragments 11P2 and 11P23 are located next to each other.

E. coli XL1 Blue MRF′ cells were cultivated overnight at 37° C. in 5 ml of LB-medium, supplemented with 50 μg/ml of kanamycin. For sequencing reactions the plasmids were isolated using alkali lysis method described by Sambrook et al. (1989), and purified using Qiaquick Gel Extraction Kit from Qiagen, or the plasmids were isolated using Wizard Plus Minipreps DNA Purification System kit (Promega) according to the manufacturer's instructions. DNA sequencing was performed using the automatic ABI DNA sequencer (Perkin-Elmer) according to the manufacturer's instructions.

1.4 Sequence Analysis and the Deduced Functions of the Genes

Sequence analyses were effected using the GCG sequence analysis software package (Version 8; Genetics Computer Group, Madison, Wis., USA). The translation table was modified to accept also GTG as a start codon. Codon usage was analysed using published data (Wright and Bibb, 1992).

According to the CODONPREFERENCE program the sequenced DNA fragment contained 17 complete open reading frames (ORFs), as well as one 3′ end and one 5′ end of two other ORFs. The functions of the genes were concluded by comparing the amino acid sequences translated from their base sequences to the known sequences in data banks. The results are shown in the following Table 1 referring to the sequence data given in the application.

TABLE 1

| | | Gene | Position | Homology %/ | Accession |
|---|---|---|---|---|---|
| | Putative function | product | (SEQ ID NO) | Similarity % | number |
| Polyketide | minPKS KSI | OrfA | 13907-15142 | UrdA (80/88) | CAA60569 |
| synthesis | | | compl (2) | | |
| | KSII | OrfB | 12681-13910 | S. venezuelae | AAB36563 |
| | | | compl (3) | chain length determinant | |
| | | | | (71/80) | |
| | ACP | OrfC | 12421-12684 | S. venezuelae | AAB36564 |
| | | | compl (4) | acyl carrier protein (61/71) | |
| | ketoreductase | OrfD | 11570-12352 | S. venezuelae | AAB36565 |
| | | | compl (5) | ketoreductase (80/90) | |
| | oxygenase II | OrfE | 15586-17055 | UrdE (68/77) | CAA60567 |
| | | | compl (6) | | |
| | cyclase | OrfF | 15208-15537 | LanF (77/85) | AAD13535 |
| | | | compl (7) | | |
| | cyclase | OrfL | 10557-11504 | gris ORF4 (72/82) | E55587 |
| | | | compl (8) | | |
| | oxygenase I | OrfM | 9014-10555 | LanM (62/73) | AAD13541 |
| | | | compl (9) | | |
| | reductase I | OrfV | 8178-8939 | UrdM (73/83) | AAF00206 |
| | | | compl (10) | | |
| | reductase II | OrfO | 4854-5438 | LanO (63/72) | AAD13543 |
| | | | compl (11) | | |
| Glycosylation | dTDP-glucose-4,6- | OrfH | 2712-3710 | LanH (71/82) | AAD13546 |
| | dehydratase | | compl (12) | | |
| | NDP-hexose-3- | OrfQ | 1391-2701 | UrdQ (83/91) | AAF72550 |
| | dehydratase | | compl (13) | | |
| | NDP-hexose-2,3- | OrfS* | -561 compl | LanS (68/74) | AAD13549 |
| | dehydratase | | (14) | | |
| | 4-ketoreductase | OrfR | 580-1335 compl | LanR (66/77) | AAD13548 |
| | | | (15) | | |
| | O-acyltransferase | OrfY | 5494-6687 compl | MegY (42/58) | AAG13909 |
| | | | (16) | | |
| Regulation | regulation | OrfR1* | 18603-compl (17) | JadR1 (58/70) | AAB36584 |
| Resistance | transporter | OrfJ | 6780-8051 compl | UrdJ2 (51/61) | AAF00207 |
| | | | (18) | | |
| Unclear | unknown | Orf1 | 17692-18492 | S. fradiae | AAD40806 |
| | | | (19) | ORF12 (41/49) | |
| | homologous to | Orf2 | 3793-4848 | S. coelicolor | CAB72221 |
| | oxidoreductases | | compl (20) | SCE56.02 (40/55) | |

*Partial sequence compl = complementary sequence

1.5 Expression Cloning

The two 9.5 kb PstI fragments were cloned into the plasmid pIJ486, and designated as pS11P2 and pS11P23. The plasmids were introduced into the S. lividans strain TK24, isolated from it and introduced further to S. argillaceus and then first into S. galilaeus mutant H039, and then into S. galilaeus mutant H075.

### EXAMPLE 2

### Compounds Generated by 11P2 and 11P23 Clusters

2.1 Cultivation and Purification

According to the initial HPLC-DAD analysis, the strains TK24/pS11P2, HO39/pS11P2 and S. argillaceus/pS11P2 produced unknown compounds, together with known compounds related to corresponding parent strains. Each strain was fermented at 10 l scale for purification and identification of unknown products. After seven days' fermentation (El medium, 28° C., 300 rpm, aeration 10 l/min) the mycelia were separated with ultrafiltration. Prior to the separation the pH of the broth was adjusted to be between 4 and 5. The mycelia were extracted three times with methanol (3×1l). The supernatant was treated for 30 min with 300 g of Amberlite XAD-7 resin, which was collected and subsequently extracted with 2 1 of methanol. Combined methanol extracts were vacuum-concentrated to 200 ml.

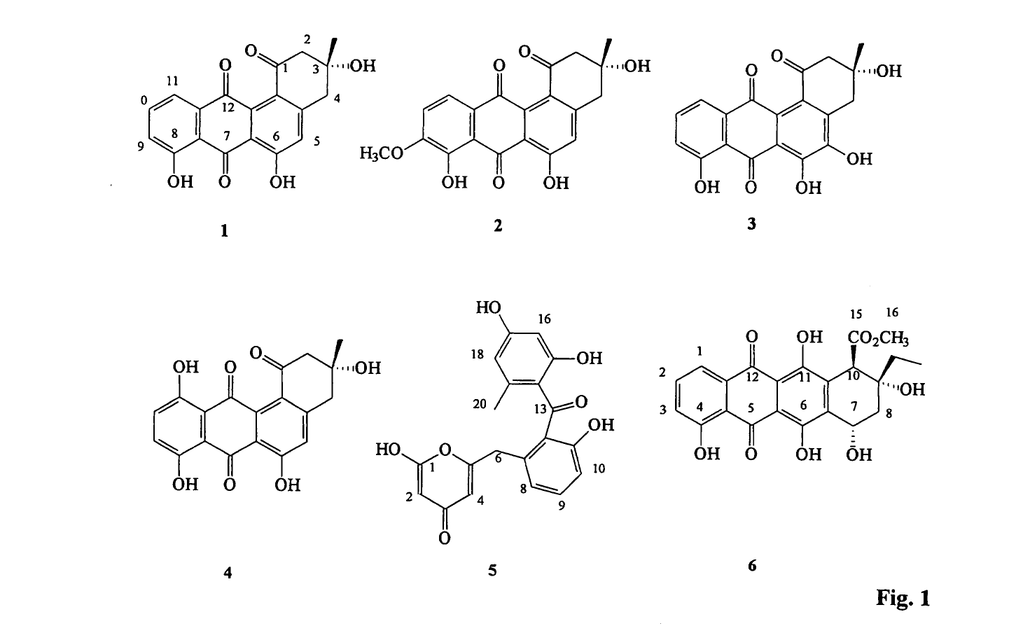

The liquid residue was loaded onto a RP-18 flash column (5×6 cm) and eluted with a descending gradient of methanol/water, starting from 70% of water. Fractions were analysed by TLC and pooled based on analysis. Pooled fractions were extracted with chloroform, washed with water and concentrated to dryness. The dry residue was loaded onto a SiO₂ flash column (2×10 cm) loaded with dichloromethane. The column was developed by increasing stepwise the portion of methanol in dichloromethane up to 25%. Fractions were detected with TLC and pooled according to analysis. Pooled fractions were evaporated to dryness and applied to preparative HPLC (RP-18, 250×10) using a descending gradient of acetonitrile-0.1% HCOOH eluent. Pooled pure fractions were extracted with chloroform and dried for spectroscopic evaluation. The production levels of the products (1-6, FIG. 1) in corresponding strains were below 10 mg/l.

2.2 Identification

Identification of the compounds was based on unambiguous assignation of carbon and proton resonances using a standard combination of HMBC, HSQC, TOCSY and NOESY experiments. The results are depicted in Tables 2 to 4 below. The results were also confirmed with mass spectrometric (MS) data from compounds (1)-(3), giving the correct molecular mass for each, and expected degradation patterns consistent with the structures. The structures of the compounds (4)-(6) were deduced from NMR-data.

MS Results for Compounds (1)-(3):

(1) EIMS, m/z (relative intensity): 338(M⁺/10), 320(35), 310(45), 295(15), 280(100)

(2) EIMS, m/z (relative intensity): 368(M⁺/15), 350(100), 310(25), 279(15)

(3) EIMS, m/z (relative intensity): 354(M⁺/5), 336(100), 326(7), 311(7), 296(10)

TABLE 2
¹³C data (δ, multiplicity) for compounds (1)-(4)
at 100 MHz in CDCl₃ (1)-(2) and in 1:1 mixture
of CDCl₃ and d₆-DMSO (3)-(4).

| | | 9-OMe- | 5-OH- | 11-OH- |
|---|---|---|---|---|
| | rabelomycin | rabelomycin | rabelomycin | rabelomycin |
| Site | (1) | (2) | (3) | (4) |
| 1 | 196.6(s) | 196.0(s) | 196.5(s) | 196.6(s) |
| 2 | 54.1(t) | 53.1(t) | 53.2(t) | 51.9(t) |
| 3 | 70.9(s) | 71.0(s) | 70.9(s) | 71.1(s) |
| 4 | 36.9(t) | 43.0(t) | 37.8(t) | 38.2(t) |
| 4a | 150.6(s) | 151.2(s) | 135.7(s) | 150.0(s) |
| 5 | 122.2(d) | 121.0(d) | 147.8(s) | 120.6(d) |
| 6 | 160.2(s) | 162.1(s) | 150.6(s) | 161.6(s) |
| 6a | 115.9(s) | 116.6(s) | 115.9(s) | 115.2(s) |
| 7 | 192.2(s) | 192.8(s) | 192.4(s) | 192.2(s) |
| 7a | 115.4(s) | 115.4(s) | 115.2(s) | 115.0(s) |
| 8 | 160.9(s) | 151.2(s) | 161.1(s) | 161.0(s) |
| 9 | 120.6(d) | 153.0(s) | 122.9(d) | 120.8(d) |
| 10 | 135.8(d) | 117.8(d) | 137.5(d) | 119.6(d) |
| 11 | 119.0(d) | 120.2(d) | 118.8(d) | 160.3(s) |
| 11a | 135.2(s) | 126.2(s) | 135.8(s) | 116.8(s) |
| 12 | 181.8(s) | 181.8(s) | 181.5(s) | 188.6(s) |
| 12a | 129.2(s) | 137.0(s) | 126.2(s) | 125.9(s) |
| 12b | 132.0(s) | 129.9(s) | 130.7(s) | 130.0(s) |
| 13 | 29.4(q) | 29.0(q) | 29.1(q) | 28.9(q) |
| 9-OMe | — | 55.2 | — | — |

TABLE 3
¹H data (δ, multiplicity, Jₕₕ, area) for compounds (1)-(4)
at 400 MHz in CDCl_{3 (1)-(2) and in 1:1 mixture of}
CDCl₃ and d₆-DMSO (3)-(4).

| | | 9-OMe- | 5-OH- | 11-OH- |
|---|---|---|---|---|
| | rabelomycin | rabelomycin | rabelomycin | rabelomycin |
| Site | (1) | (2) | (3) | (4) |
| 2a | 3.01, d, 15.1, 1H | 2.85, d, 14.4, 1H | 2.92, d, 13.6, 1H | 3.00, d, 15.0, 1H |
| 2b | 2.95, d, 15.2, 1H | 2.75, d, 14.4, 1H | 2.74, dd, 13.6, | 2.93, d, 15.1, 1H |
| | | | 1.2, 1H | |
| 3-OH | exchange | exchange | exchange | exchange |
| 4a | 3.08, brs, 2H | 2.98, brs, 2H | 3.15, dd, 17.6, | 3.01, brs, 2H |
| | | | 1.2, 1H | |
| 4b | — | — | 2.83, d, 17.6, 1H | — |
| 5 | 6.99, s, 1H | 6.94, s, 1H | — | 6.98, s, 1H |
| 5-OH | — | — | exchange | — |
| 6-OH | 12.22, s, 1H | 12.00, brs, 1H | exchange | 12.47, s, 1H |
| 8-OH | 11.65, s, 1H | 11.96, brs, 1H | exchange | 11.95, s, 1H |
| 9 | 7.26, d, 7.6, 1H | — | 7.26, dd, 8.2, | 7.25, d, 9.3, 1H |
| | | | 1.5, 1H | |
| 9-OMe | — | 3.91, s, 3H | — | — |
| 10 | 7.65, dd, 8.1, | 7.22, d, 8.0, 1H | 7.74, dd, 8.2, | 7.20, d, 9.3, 1H |
| | 7.6, 1H | | 7.6, 1H | |
| 11 | 7.25, d, 8.1, 1H | 7.52, d, 8.0, 1H | 7.51, dd, 7.6, | — |
| | | | 1.4, 1H | |
| 11-OH | — | — | — | 12.16, s, 1H |
| 13 | 1.49, s, 3H | 1.31, s, 3H | 1.37, s, 3H | 1.44, s, 3H |

TABLE 4
¹H (δ, multiplicity, Jₕₕ, area) and ¹³C (δ, multiplicity)
spectral data for compounds (5) and (6)
at 400 and 100 MHz, respectively, in CDCl₃ (5)
and in d₆-DMSO (6).

| | 19-methyl-SEK15 (5) | | ε-rhodomycinone (6) | |
|---|---|---|---|---|
| Site | ¹³C | ¹H | ¹³C | ¹H |
| 1 | 163.5(s) | — | 119.7(d) | 7.75, d, |
| | | | | 8.3, 1H |
| 1-OH | — | 11.55, brs, 1H | — | — |
| 2 | 88.2(d) | 5.13, d, 1.9, 1H | 137.2(d) | 7.61, dd, 8.3, |
| | | | | 7.7, 1H |
| 3 | 170.2(s) | — | 124.9(d) | 7.22, d, |
| | | | | 7.7, 1H |
| 4 | 101.1(d) | 5.68, d, 2.0, 1H | 162.9(s) | — |
| 4-OH | — | — | — | 11.95, s, 1H |
| 4a | — | — | 115.9(s) | — |
| 5 | 163.7(s) | — | 190.8(s) | — |
| 5a | — | — | 111.2(s) | — |
| 6 | 36.4(t) | 3.57, s, 2H | 155.9(s) | — |
| 6-OH | — | — | — | 13.33, s, 1H |
| 6a | — | — | 137.5(s) | — |
| 7 | 132.6(s) | — | 62.6(d) | 5.25, brs, 1H |
| 8 | 121.0(d) | 6.75, dd, 8.1, 1.0, 1H | 34.4(t) | 2.19, cm, 2H |
| 9 | 130.1(d) | 7.21, dd, 8.1, 7.8, 1H | 71.4(s) | — |
| 10 | 114.6(d) | 6.78, dd, 7.8, 1.1, 1H | 51.5(d) | 4.18, s, 1H |
| 10a | — | — | 135.0(s) | — |
| 11 | 153.8(s) | — | 157.0(s) | — |
| 11a | — | — | 111.4(s) | — |
| 11-OH | — | 9.78, s, 1H | — | 12.77, s, 1H |
| 12 | 130.8(s) | — | 186.0(s) | — |
| 12a | — | — | 133.3(s) | — |
| 13A | 199.9(s) | — | 32.6(t) | 1.71, dq, 14.3, |
| | | | | 6.3, 1H |
| 13B | — | — | — | 1.45, dq, 14.3, |
| | | | | 6.2, 1H |
| 14 | 115.6(s) | — | 6.8(q) | 1.08, t, 6.3, 3H |
| 15 | 165.1(s) | — | 171.3(s) | — |
| 15-OH | — | 12.67, s, 1H | — | — |
| 16 | 100.7(d) | 6.12, d, 8.3, 1H | 52.4(q) | 3.65, s, 3H |
| 17 | 163.2(s) | — | — | — |
| 17-OH | — | 10.41, brs, 1H | — | — |
| 18 | 111.6(d) | 6.08, d, 8.3, 1H | — | — |
| 19 | 143.0(s) | — | — | — |
| 20 | 21.5(q) | 1.83, s, 3H | — | — |

Deposited Microorganisms

The following microorganisms were deposited in Deutsche Sammlung von Mikro-organismen und Zellkulturen (DSMZ), Mascheroder Weg 1 b, D-38124 Braunschweig, Germany.

| | Accession | |
|---|---|---|
| Microorganism | number | Deposition date |
| Streptomyces lividans TK24/pS11P2 | DSM 14172 | 13 Mar. 2001 |
| Streptomyces lividans TK24/pS11P23 | DSM 14173 | 13 Mar. 2001 |

Sequence Listing Free Text

For:
SEQ ID NO:2 “translate of OrfA, putative function: ketosynthase I”
SEQ ID NO:3 “translate of OrfB, putative function: ketosynthase II”
SEQ ID NO:4 “translate of OrfC, putative function: acyl carrier protein”
SEQ ID NO:5 “translate of OrfD, putative function: ketoreductase”
SEQ ID NO:6 “translate of OrfE, putative function: oxygenase II”
SEQ ID NO:7 “translate of OrfF, putative function: cyclase”
SEQ ID NO:8 “translate of OrfL, putative function: cyclase”
SEQ ID NO:9 “translate of OrfM, putative function: oxygenase I”
SEQ ID NO: 10 “translate of OrfV, putative function: reductase I”
SEQ ID NO: 11 “translate of OrfO, putative function: reductase II”
SEQ ID NO: 12 “translate of OrfH, putative function: dTDP-glucose-4,6-dehydratase”
SEQ ID NO: 13 “translate of OrfQ, putative function: NDP-hexose-3-dehydratase”
SEQ ID NO: 14 “translate of OrfS, putative function: NDP-hexose-2,3-dehydratase”
SEQ ID NO: 15 “translate of OrfR, putative function: 4-ketoreductase”
SEQ ID NO: 16 “translate of OrfY, putative function: O-acyltransferase”
SEQ ID NO: 17 “translate of OrfR1, putative function: regulation”
SEQ ID NO: 18 “translate of OrfJ, putative function: transporter”
SEQ ID NO:19 “translate of Orf1, putative function: unknown”
SEQ ID NO:20 “translate of Orf2, putative function: oxidoreductase”
SEQ ID NO:21 Description of Artificial Sequence: oligonucleotide primer
SEQ ID NO:22 Description of Artificial Sequence: oligonucleotide primer
   References
Dairi, T., Hamano, Y., Furumai, T. and Oki, T. (1999). Development of a self-cloning system for Actinomadura verrucosospora and identification of polyketide synthase genes essential for production of the angucyclic antibiotic pradimicin. Appl. Environ. Microbiol. 65:2703-2709.
Decker, H. and Haag, S. (1995). Cloning and characterization of a polyketide synthase gene from Streptomyces fradiae Tü2717, which carries the genes for biosynthesis of the angucycline antibiotic urdamycin A and a gene probably involved in its oxygenation. J. Bacteriol. 177:6126-6136.
Gould, J., Hong, S. and Carney, J. (1998). Cloning and heterologous expression of genes from the kinamycin biosynthetic pathway of Streptomyces murayamaensis. J. Antibiot. (Tokyo) 51:52-57.
Han, L., Yang, K., Ramalingam, E., Mosher, R. and Vining, L. (1994). Cloning and characterization of polyketide synthase genes for jadomycin B biosynthesis in Streptomyces venezulae ISP5230. Microbiology 140:3379-3389.
Hong, S., Carney, J. and Bould, S. (1997). Cloning and heterologous expression of the entire gene clusters for PD 116740 from Streptomyces strain WP 4669 and tetrangulol and tetrangomycin from Streptomyces rimosus NRRL 3016. J. Bacteriol. 179:470-476.
Hopwood, D., Bibb, M., Chater, K., Keiser, T., Bruton, C., Kieser, H., Lydiate, D., Smith, C., Ward, J., and Schrempf, H. (1985). Genetic manipulation of Streptomyces: a laboratory manual. The John Innes Foundation, Norwich, United Kingdom.
Krohn, K. and Rohr, J. (1997) Angucyclines: total syntheses, new structures, and biosynthetic studies of an emerging new class of antibiotics. Top. Curr. Chem. 188:127-195.
Metsä-Ketelä, M., Salo, V., Halo, L., Hautala, A., Hakala, J., Mäntsälä, P. and Ylihonko, K. (1999). An efficient approach for screening minimal PKS genes from Streptomyces. FEMS Microbiol Lett. 180:1-6.
Oki, Toshikazu, Dairi and Tohru, WO 98/11230. Polyketide synthases of Actinomadura involved in pradimicin biosynthesis and the genes encoding them. (Bristol-Myers Squibb Company, USA).
Rohr, J. and Thiericke, R. (1992). Angucycline group antibiotics. Nat. Prod Rep., 9:103-137.
Sambrook, J., Fritsch, E. and Maniatis, T. (1989). Molecular cloning: a laboratory manual, 2nd ed. Cold Spring Harbor Laboratory Press, Cold Spring Harbor, N.Y.
Ward, J. M., Janssen, G. R., Kieser, T., Bibb, M. J., Buttner, M. J. and Bibb, M. J. (1986). Construction and characterization of a series of multicopy promoter-probe plasmid vectors for Streptomyces using the aminoglycoside phosphotransferase from Tn5 as indicator. Mol. Gen. Genet. 203:468-478.
Westrich, L., Domann, S., Faust, B., Bedford, D., Hopwood, D. A. and Bechthold, A. (1999). Cloning and characterization of a gene cluster from Streptomyces cyanogenus S136 probably involved in landomycin biosynthesis. FEMS Microbiol. Lett. 170:381-387
Wright, F. and Bibb, M. (1992). Codon usage in the G+C-rich Streptomyces genome. Gene. 113:55-65.
Ylihonko, K., Hakala, J., Niemi, J., Lundell, J. and Mäntsalä, P. (1994). Isolation and characterization of aclacinomycin A-nonproducing Streptomyces galilaeus (ATCC 31615) mutants. Microbiol. 140:1359-1365.

1. An isolated and purified DNA fragment, which is the gene cluster for rabelomycin biosynthetic pathway of Streptomyces bacteria, being included in two 9.5 kb flanked PstI fragments of Streptomyces sp. genome.

2. The DNA fragment according to claim 1, comprising the nucleotide sequence given in SEQ ID NO:1, or a sequence showing at least 90% homology to said sequence.

3. A recombinant DNA, which comprises the DNA fragment according to claim 1, or any one of the two 9.5 kb PstI fragments thereof as defined in claim 1, cloned in a plasmid replicating in Streptomyces.

4. The recombinant DNA according to claim 3, which is the plasmid pS11P2, deposited in S. lividans strain TK24/pS11P2 with the accession number DSM 14172.

5. The recombinant DNA according to claim 3, which is the plasmid pS11P23, deposited in S. lividans strain TK24/pS11P23 with the accession number DSM 14173.

6. A process for the production of hybrid polyketide compounds, comprising transferring the DNA fragment according to claim 1 into a Streptomyces host, cultivating the recombinant strain obtained, and isolating the compounds produced.

7. The process according to claim 6, wherein the Streptomyces host is a Streptomyces lividans host.

8. The process according to claim 6, wherein the Streptomyces host is a Streptomyces argillaceus host.

9. The process according to claim 6, wherein the Streptomyces host is a Streptomyces galilaeus host.

10. The process according to claim 6, wherein an angucycline is produced, which has the following formula (2)

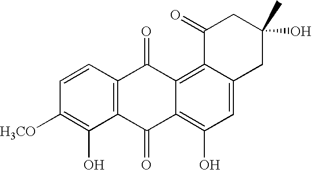

11. The process according to claim 6, wherein an angucycline is produced, which has the following formula (4)

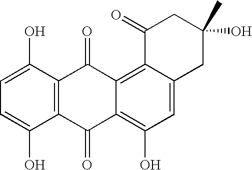

12. A process for the production of hybrid polyketide compounds, comprising transferring at least one of the genes selected from the group consisting of Orfs A, B, C, D, E, F, L, M, V, O, H, Q, R, Y, J, 1 and 2 into a Streptomyces host, said genes being derived from the DNA fragment according to claim 1, cultivating the recombinant strain obtained, and isolating the compounds produced.

13. A process according to claim 12 for generating novel compounds for drug screening.

14. An angucycline compound, 9-OMe-rabelomycin, which has the following formula (2)

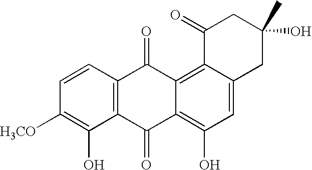

15. An angucycline compound, 11-OH-rabelomycin, which has the following formula (4) [chemical structure]


SEQUENCE LISTING (22 sequences)

SEQ ID NO: 1 - DNA, 19016 bp, Streptomyces sp. H021
ctgcagcacgtcggcgacgacgctgccggcttcagggctgacgaagtggtcgatgtaccggacgggcgcgccccggtgggcgcccgtgtagttgctgcgggtcgcctggacggtcggcgagagctggaggaggttggggttgccgggctccatcttggcctgcatcaggaagtgcgggacgccgtcgaactccttgaccagcagccccaggatgccgacctcgggctgtttgatgatcggctggcgccaggtcgtcacggtcccgtcgtcgtcggtgaccgtggcgtccagtccctcgacggagaagaaccttccgctgctgtggacgaggttgccggtgtcgggggcgaagtcccagccgcggagttcggcgaacgggatgcggtcgacccggaagtcgtgggcacgcctgcgttcctccagccaggccggcacgtcggcggtgcggatcgccgccccttcgagggcctcggcggaccgggcgtaccgctcgacggtcgcggcctcccggcgcggccggagaacggggtgggtgagcgtggttgcggacagggcgggcatggtgggccaaccttcgggtcagggcgtcgccgtggggacggccgccgtcagaaaggcgtcgagcacccggcggtggtagctcgggccgaagcccatggcctcggtctcgggcaccagggcgcgcagcttgtcggtggagatggtgtgatgtccgccccggtcccggtacacccggcgcgcggcgaggccggtgcgctcctcgatgtggtcgatgatccgctcgatcggcacggcgttgccggaggcgacgttgaccttctcccggctgaggcccgcgccgagcagacggtcgacgatggtgacgacgtcgtcgatgtcgatgaggtcgcgggtggcgccccggtggacgtggaccgttccggtgcgcatctggcgcacgagggtcggcagcagttggtgcggtggctgccccggtccgacgaggtgtcccaggcgcagggccaggtagtcgacgccggaggcctccagccgtttctccagcgccagcttgtgcgtgccgtacggggtgcaggggacgacggggcggtcctcgcggccggggccgtccacctttccgtacatgccggtggaggcggtggagaagaacaccagacggtcgccggaggccaggcaggcggcgatcttctcctccaccagggcggcttcgcgggcgaagtcggccggcgaggtgtgcgcggcccaggacacgcctgcggcgagcaccagcgtgcctccgtggcgcccggccagcgaacgcaggtgccgggcgaggaatccgtttcccactatgtccatgtcggtcctgatcgctggggcggtcgggttcgggccgccccacgggggcgggcgatcatccgcgggcctgtacgaactcccggatcgacgcgacgacgtagtcgatcatttcgtccgtgagggcgggatagaccccgatccagaaggtctggtcggtgatgaggtcgctgttggtcagctcgcccaccacgcgctgcggctgcccgatgtaggcggggtggcgggtgaggttgccggcgaagagccgcctggtgccgatcttgcggctctccaggaagtcgaccgtctcggccctgctgaagggggcctcgggatcgatcgtcaggacgaagccgaaccagctcgggtcgctgcgcggggtggcctcgggcagcaccaggtggggtacgtccgccagcccgtcgcgcagccgccgccagttgcggcggcgcgccgcgcagaactcgtccagcttggccagctggctgaggcccagggcggcctggaggtcggtcgccttcaggttgtacccgacgtggctgaagatgtacttgtggtcgtagccgacggggagcgttcccatctggtactcgaaccgcttgagacattttccgctctcgcccggctcgcaccagcagtcgcggccccagtcgcgcagggactccacgatgcgtgccagcgccaggttggcggtcagcacgcagccgccctcgcccatcgtcaggtggtgtgcggggtagaagctgacggtggacaggtctccgaaggtgccggtgagctgcccgtcgtagagggacccgacggcgtcgcagttgtcctcgatgaggaacaggtcgtggtcggccgccagttgggcgatctcggtggcctcgaaggggttgccgagggcgtgcgcgatgatgatcgccctggtgcgcgggccgatggcccgggccacgcggtcggcggtggtgttgtaggtgccgatctccacgtcgacgaagaccgggatcagcccgttctggaggatcgggttgacggtggtggggaagccggccgcgacggtgatgacctcgtcccccggccgcagccgccggtcctccagcaggtgcgaggtgagcgccgagaccgagagcaggttcgcggacgaccccgagttggtcagatgcgccttgcggcgtcccatcctgcgggcgaaggcggactcgaatctgcgcgaactcactcccgcggcgatgcgcatgtccagcgccgcctcggcgagcgccgctcgatcgtcctcgtccagtaccgcgccggagggccagatctcggtgacgcccggcacgaaccgccggtccggctgtgcctcgtggtggtacttgcgtacctcgtccagcaacagcgccttgcgatcgagggggtccactaggtctccttcaggtcaggacgctgaggtccgggcgctcggccacggccgccgcgtcctgccaccagtcggtgtgctcccggtaccaggcgactgtctcggccagcccctgatcgaacgtgatctgcggcgcgtaccccagctcctcgcggatcttggtgtcgtccagcgagtagcgcaggtcgtgccccttgcggtcggccaccctccgcaccagcgactcgtcggcgccgcagaggtcgagcagccgtccggtgaggtccaggttggtccactcgttgccgccgccgacgttgtacgtctcgcccgagcggcccttgttcagcacgaggtggagcgcccggcagtgatcgtccacgtgcagccactcgcgcatgttgccgccgtccccgtacagcggcaccggcatgccgcgcagcagccgggtcgcgaacagcggaatcagcttctcggggtgctggtgcggaccgtagttgttggagcagcgggtgaccgagacgtccaggccgtgggtgcgccagtagctgcgggccaccaggtcggaggccgccttggacgccgcgtagggcgtgttgggggccagcggccggtcctcggtccaggacccctcggagatcgatccgtacacctcgtcggtggagacgtggacgacgcgttccacgtccgcgcgcagcgccgcttcgagtacggactgcgtgcccagcacattggtgcggtagaactcggcggggccggtgagggagcggtccacatgggactcggcggcgaagtgcaccaccgcgtggactccgtcggccagctcccgcacggtctccgtgtcgcacacgtcgccccgtacgaagcgcaggcgcggatgggacgcgggcaggttggcgcggttgcccgcgtaggtgagggcgtccaggacgacgacctcggcgtcctcccagccctcgaaccggttctccaacagcatcctgacgaagcgggagccgatgaaaccggcgccgccggtgaccagcattctcatgtctcgctctccttccgggtacgtccttggggtacgtgcgtgccgcgcccgcgtggtccctccgtcgcaccgggccgtcgcgtcacaggagggtggcgaccgtgcgtcccggtccccggcggcgttccaggtccggccagcggtcgctgtaggcgccctgggtgaacttgccgtggcgcatcagctccctggggaagccgagggagacctcgctgagggagtcgagccgccgggtctgctcctcgtcgagccggacgtcgagagcggcgagggagtcctgaagctgtccgggccgggtcgcgccgaggatcggcaggacggcctcgggccgggcgcgcagccaggcgagtgcgacctgcgcgggggtccagccgccttcctcggcgacggccacgacctcccgtacgacggtggtgtcgtgctcgtcgtcgcccgcccagttctccaccgtgagccggccgctctccccgcgcagatacttgccggtcagccgcccctcggccagcggaccccaggcgaaggcgctgacgtcgcaggcggaggccatcgggagcaggtcccgttcggcggtgcgttccagcaggttgtagcggacctggaggcccgcgaacgccgaccagccgcgcagctcggcgagcatgttggcgcgggagacctcccaggcgggccagttggaggcccccacgtagaggaccttgccggcccggatctggtcgtcgagggcgcgcatgacctcctcgacgggggtgaacgcgtcccgggcgtgcagccagagcacgtccacccggtcggtccgcagccgccgcaggctgttctccagcgaggcgacgaggttcttgcggtggttgcccgccgagttcacgtcgcccgcccgggtcatggtgttgtacttggtggccaggacgaactcgtcgcgtcggcccgccagtacctcgccgaggcactcctccgagcgtcccccgccgtagacgttcgcggtgtcgatgaagttgccgcccgcctgcgcgtagcggtcgaccatgagcaaggcgtcctccttcgaggcgccccacccctcgaccccgaaggtcccggcgcccaggcagagttcggagacccgtacgccggtcctgccgaggaggtcgtacttcatgctcacgccgttcactccccgtacggctggtcggcgcgcgcctgccgcagggcccgcccccaccatccgagctggtccagcaggaccttggcggcggctccgcagctctccgggtcgcgggggtggggcccgtcgaaggtggcggcgccgccgtggaagctgacggtgtcgcggaccgtgacggcgtggagttccgcgaagacctggcgcagttgttccaccgcgcgcaggccgccggcgatgccgccgtacgacacgaagccgaccggcttcgcggcccactcggtgaggtgccagtcgatgacgttcttcagggccgccgggaagctgtggttgtactcgggggtgacgacgacataggcgtcggcgcgctggaaccggggccgggaccgcgcgagcagggcggtcatgtcggccgtgggggtgcgactgggcccggccaggtcgaagggcagctcggccaggtcgatcacgtcgagggtgaggtcggggcgctgggcggccagctcggcgaaccaggcggcgacggtgtgaccgaagcgttcggtcctgacgctgccgatcagcacggcgatatggaggggctgctgcgacatgcgagtccttgtcgggtgtgtggggcggtgcggtcggtggtcgggggccgcgtcctcagtgggccgcgggggcttcggcggcccgttccggccggacggccgtctgctcggtgcgccggggtcccggcgggcggccccagcggcgcatgacggggcgttcgacgatcgcgtacagggcccacgcggcgagcacggacacgacgagggccgccgccaggaagagcaggccgccgggtgtgctccaggcgggtccggagggcttgccgaagatgttggggtccgagccgaacagccggtgaccgtagatcaggatcagatagtgcacgcagtagaaggcgaacgagatctccccgagccagaccatggggcgcttgctcagcaagctctcgcggcccttggcgtcggaggcggcggcggccgggatcagcagggccagcgggacgacggtgatcgccacgtagttgtagagcgggttgtggtcgatcagtacggcccccacgtaggcggcgatcgtcaggagggccggcggtatcacgccgagcccgatccaccggccggacagcacgatgcgcgccagcagcatgccgagcacgaattcgaggagccgggcgacggggaagacgtacacgaaccagatctgggtccaggagatcttcatgaacatgaagcccggggtgtccggcagcagcacctgcgcgatcagcggcatcagcatgatgaccagcacgacgccacccgccgcgtaccagagcacggccggcgcgatccggctcacgcccttgatgagcagcggaaaggacaggtagaagaggagctccacggagagcgaccagctgacggtgttggccgtttcgacgtagccgctgtcgggcacccaggcgtgcaccaggaacaggttggcgagcgtctcggggaaggcgaccgcggtactggtcaccgtcagcaggacgatggccacgacgaacgtcaccaggtggttggggaagatcttcagcaggcgccgccgccagaatccgcggacggtgtcggcgggacgcaccgaccaggtgaggacgaagccgctgagcatgaagaagaaggagacgccgaccgcgccggcgttgccgaaccagcggtagtagtcggctgcgaccccctggtccttgaagacgccggcggcggccccgtgcacggagaacaccagcagggcggcgagaaaacgcatcccggtgagcgaggggagtctggaggtggcgttcgatggcgccatggcacgtctgccttactcggtcgcgacgaccggggaacgggggcggaagggaggcgggacgccccctggggaggggcggcgggcggtacgggtcagcggtcgtcggcgacggccgggcgacgggcggtgaggccgcgcagggcgaccagcaggcccagcgccgcgagggcgcagagcaggagcaggacgggccggtgggcggcgtcccccaccgggccaccgtccccgggcccgaaggtgagcagcgcggccacggcggcgggcaccgcgaccgctccggcctggaccgccgtctggtaggtggcggtggccgtcccccggtcggccgtcccgacggcccggcccgcctgcgcgttcagcgccgcgaaggccggtacgaagccgaggccgaccagtgccagcgtcggcagcaggtccgtggcgtaggaccgcggcaggtcgaggcgtgcgtacagcagcaggcccacgaggtggagcgccgcgccgaggacgacgagccggggggcgccgaaccgggccaccacacgcccggccgagagcgctgtcaccgccaggggcaggcaggccgggaggaaggcggccgcggtctgccagggactccagtgcagcaggtcccaggcctggtgggccatcagggacagcagccccagataggtgccgttgaggatcgccgcaccgagcgccggacgcagcagcgctccgttgcccagcagtcggcgcaccgtcggagcgatcggctgcgcggaccccggctgttgccgggggacggccgcggccgccaggacgagcagcaccgcggcgaccggggccgtggcgacgaaggtccagtgccagtcgtgcggtgcgagggctcccgccagaagcaggcccgtggtgaatccgatgccgccgcagaaggtgtacacggacagggcacgggcgcgcgccgaggcttccccgtaggtggtggagatgatggcgaggccggtgggcgcggtcagggcggcgcacatgcccttgacgatccggctgcccaggaggagcagcgggtcgtccgtgagtccgccggccatcgaggcgaggacgaagagggccagcgcccccaggtacagccggcggtgtccgaaccgcgccaccaggagccggcccggcaggagcagggccccgaagccgagcgcgaacccgcccatggcccactggacctgccagggggacagcccgaggtcggcgccgatcgacggcagggcgacgacgaccacggacacttcgagaccgtcgatgagcatgttgccggcgaggacgagcagcaggaagcgggctgcccacggccggggcggggcgccgggcgacgggccggtcttcgaggggacggggggccgcgacatcgggtgttcaccgccttggattcgggacgactcggttgcggatgcccggggttcggaccgcgcgggtccggaccacgtgggctcggaccgctcgggttcggaccgctcgggttcggaccgcgtgggtcagccgaggagtgtgccgccggtggcgtcgatgaaggagccggtgatccagcgggcgtcgtcggaggcgaggaacgccaccacgtcaccgacgtccgcgggcttgcccacctgtccgaaggccgacatctgggccatctgctccaccgcctgcgggatgtcgaacaccgggctgccgttgtcggtgatcccgggggcgacgctgttcacggtgatgccccgggcgcccaggtatttggcgaagtgcagggcgatctgttcgaccgcgcccttggtcatcgcgtaggcggcctcttcggggttggcgaaccgggtgagcccggaggagatgttgatgatgcggccgccgtcggcgagcagtccgagcgcgcgctggacgatgaagaacggcgccttcgcgttgatggcgaagagccggtcgaacagctcgggtgtgacctcctccggggggacgccgcccatgatggccgcgttgttgaccaggatgtcgagggtggcgaccccggtccgttccttcagtccgctctccagcgcctcgaacagcgtgtcgatgtcgccggggacgccgaactcgctgcgaacggcgaacgctcggccgcccgccgcctcgatgtcggcgaccgtgctcagcgcggcgtcctcgccggtggcgtagtgcacggccaccagtgcgccctcatgggcgagccgcagtgcggtcgcccgtccgatgccccggctcgacccggtgacgagtgcggtcttgttcgcgagctttcccacggttcctccagtagtcgtcaggtcgttcttgcgatggccgtccgccgacgggcccgtccggtggtcgcgccgtgctaccggcgcgccgccgggccgaaccagcgccgcagggccgcctccagctccggaacggtgccgtcggcccaggccacatggccgtccggccgcagcagcgccgtagtgaactccggtgcggtgcagcctggttcgggcgtcgcgctcaccacgtccacccggccgtcctgccacggcgcaaccgccgcccgggccggggtcggccggccgctcaggtcgacgaggactccgcgaccggcccgcagagcggcgaagctgctgcccgggcccgcggcggtatcggtgtcgatgccggtgtcgatgccggtgtcgatgccggtgtcgaaccggaggtacgggacgcgggcgccgaccaggcggtgggcgcccggttcggccgcgtaccgtacgtcgacgcccgcgacggtcccggcgagatgggcacgcacccggggcttccgcaggagttcgccgaagagggagcgcagctgttcggtcccggagccgccgagcagcagcagcgcctgggcggcgatgttgtccagcacccgccgcccggccgggtggcgttcggcgtgatagctgtccagcagcccgggtcccgcggtcccccggacctcggcggcgagcttccagccgaggttcgcggcgtcctgcagccccaggttgagggcctggccgcccaccggcatctggtggtgggcggcgtcgcccgcgagcaggatcctgccccggcggtactgggaggcctgccggagtgcgtcgccgaaggagttcagccacaggggggctcccccgccgatgtcgtcgcccgtcacccgccgccaggtgtccgccacccgggcgaacgacgggggtccggggtcccggccggcgggggccccgtactcgtgcaccatgatccgggtgacgccgccgggcccccgggcggagatggccagtcccccggggtgacgttcgaagcgccggtcgggcacctccacgtccgccacgtccgcgcgcagcagttccctggtggcgtcccggccggggaaggcgaacccggcgagctcccggacggtgctggactcgccgtcgcagccgacgacgtacgcggcccggacgcgcagcgcggacgccgctcctccgggtcccggttccgcctcggcggtcgcctcgacggtgtgcggcgcgatcctcagatccgtgagccggtggcccttgaccacgcgggccccgagccgggtcgcccagccctccagcagttcctcggtgcggacctgggggactttccactggccggagtacgggctgtccgtggtgaggtcgagacgcgtgccgccgaagtgtccggggcccccggcgggcggggtgcccagctcctccagcaggccgcgctggtcgaggagttccatcgtgcgggcgtgcagggtggaggcccgggactcggtgagcgggccggtgcgtcgttccagcacgacgacgtcggccccgccgagggcgatttcaccggcgagcatcaggccgaccggcccggcgcccaccacgagcacctgggtgtcgatgtccggtgcgctcaccacgtcagcgccgctgtgcccgggcgtagtcgctcgcgtggccgagcgtggcgcggctgttggtgctcagcgcggagcgtacgtactcacgcgcctcctccacgcccgcgtcgggtccgagcacggcggcgatgttctccgtacggagcacgacggtgtgctgggacgtcgccacgacctccggcccgtcctgctcgaaggtccacagcccggtgtggagcgacatcagcgccggcagagtggtctgcttgtaggcgatccgttcggagggcaggcagacccggtaggactcggtcaggtgcgtcgagccgtccttggcccgggtctccatcttcaacacctgtagcccggacggctgttgcgacagctggaccgaggcgacgtgcgggagccgctcgtgccagagctgggcctcgtcgatgaagtcgtacatgtcctcggcggacccggtggcccggacggtgtcggagaaggagaaggtccacgcctcggcggcgtgcgcgttctcgacgttgtccttgagggccgcgagctcggagcgggagttgcggtccaccgcctcctcgatccagcgcagcccctccggatcgtcgtcgaccgcccggtagtcgtgcagcagccggacccgcgaccggtcggggccgagcggctccaccacccaggctccgcccatggccgccaccggcggggtggagacctcctggcggaagtcgatccgcagcccctcggggtccagggtgcggcgcgaggtccagtgcttggcctcaccgttggcggtggcccagatgcggatgcgttcccgggcgccgtcgtgctccacgcggtcgacgtggatggtcggagggaagatccggggccagttctccacctcggcgatcagccggtagacggcggaggcgggagccgagaccgtgatgtcgtgctctacctcgtgcgcaccgggtgcgggcatgcgggtgctccttccgtgaggacgtgagggggcgggccgtgaagacatcaggggggcgggccgggtcagaagttgcccaggccgccgcagacgttgagggcctgcgaggtgagcgaggcggcggtgtcggaggccagatagcccaccagtccggccacttcctcgggggtggagtagcgccccagggggatcttggcgcggaacttcgcccccaccgcctcctccgtggtttcgtacgcggccgcgtagccctgccgtacgcgctgcgccatgggcgtttcgacatagcccgggcagacggcgttgacggtgatcccggtgggggcgagctcgttgcccagggccttggtgaacccgacgacaccgtgtttggaggccgagtacggggcgccgagcaccacgccctgcttgccggccgtggaggcgatgttgatgatccggccccggctcttctcgcgcatgcctccggcccgcagcacctcacgggtcaccgtgaagacgctggtgaggttggtggcgatgacgtcgtcccacagctcgtcggcgatgtcggcggtgactccgccgccgctgcggcccgcgttgttgaccaggacgtcgatggtgccgtaccggtccaccgccgccgcgacgaaggccgcgacctcggggccggagcggacgtcgcaggccaggccgtcggcctccaggccctcggcgcgcagctccttgacggtcgaggcgacgttgtcggcgctgcgggccccgatgaagacggcgtggccctgggcgcccaggagccgggcgacggccagtccgataccgctggtgccgccggtgacgagcgcgacgcgctgctgatccggcacgtgttcctccttgtgggcgtggtccgtgaatcggtgcgtatctcaggtgcggtggggatccgtacggttcaggcggcggccgtcgccagctctccgttgacgacctccagcagggcgcggggagtcttcgaaccggtgagggagctgtcgtcgagggtgattccgtactcccgctcgatccggctgccggtctccagcagagccagcgattcgagccccaggtcctcgaacgcggcgtccagggcgtcgtcgcccaggacgtcgtcctccaggcccgccgcctcgcgcagaatgatcctgagatcgtcgaaggtgaaatgccgctgggtcatcggtctccttgtcgggttcggggggcgcgcagcaccatcgcggagttgaatccgtgatggccgcgggccaggacgagcacggtcgcgagccgcgcggggcgcgtctcgcagaccaggtcgagggcgaggccggggctcggcgccacgttgacggtgggcgggatcacgccgtccctcatggcgagcagggcgcaggccagatcgagcggtgccgctccggagtgcagccgtccggtcatcgtcttggggacggtcacgggaacgcccccggcgccgaacacctcggtcagcgccagggcctccgcccggtccaggtccggcagccccgcgccgtcggcgaacacggcgtccacgtcgtcggcggtgatgcccgcgtcccgcagggcgatctcgatcgcggtgcgcagggtcggcggccggccgctgtcgggggccgggtcgaacgtggacccgtagccggcgatctcgccgagactgcgggcgccgcgccggtgtgccgcctcctcgtcctcgacgacgaggagcgctccgccctcgccgggtacgtgtccgtgggccgcggcgtcgaaggggaggtaggcgcgcgcggggtcacgggtggtgctgagccgcccggcggacatctgcgcgacccagccccaggggcagagcgaggcgtcgatccccccggtgaggaccatggggatgcccttgcggacctggcggcgcgcctgggcgaccgcgtccaggccacccgcctgatcgctgacgaccacgctcccgggccccttcatcccgttgcggatcgatatctggccggtgttgaccgcgtagaaccaggcgaacgactggtacgcgctcacgtactgactgcccttgctccacagcgcccgcagttcgttctgcccgaactcgaagccgccagaggaactcgcggtggcgacacccatgtcgtaggcgggcgtctcctccgtccgcacgcccgcgtcggccagcgcccagtccgcgacgaccagggccagccgcgtcatccggtcggtctgcggcagcagccggcccggcagatggtcctcggcgacgaacccggggacctcgcccgcgagtccggcggggtagggcgaggcgtcgaagcggctgacgggtccgatgccgctccggccggcggtggtggccgaccagtagtcctgggtgccgaggccgttgggcgaggccacgctcaggccggagaccacgacggtcccgctcatacggctcctccttccggccgggtcagcaccatggcgctctggaacccgccgaatccgctgccgacggtgagcaccgagtcggtggaccagtcgcgtgcggtcaggggcacgtagtcgaggtcgcattcggggtcggggtggtgcaggttggcggtcggcggcacgacgtcgtactccatggcgagcgcgcacgcggcgatctcgatggagccgatggcgccgagcgagtggcccaccatggacttgatggagctgatgggcacctgccgggcgtgctcgccgagactcttcttgaaggctgccgtctcgtgccggtcgttctgccgggtgccggatccgtgggcgctgatgtagtcgatgtccgcggggtcggtgcgggcctcgtcgagggcgacgcggatggcctcggccatctccgtgccgtccggccgcagtccggtcatgtggtacgcgttgctgcgcgtcgcgtagccgctgatctccgcgtagacgtgggcgccccggtcgagggcgcttcccagctcctccaggacgaagaccgcgctgccctctcccaggacgaacccgttgcgggtggcgtcgaaagggcgggaggcgtgcgcggggtcctcgttgcgcggggtcgtcgccttgatcgcgtcgaagcaggcgagggtgatcggggagatcggggcgtccgtggctccggcgatcatgatgtccgcggagccttcccggatcagttcggtggcgtacccgacggagtcgatgcccgaggtgcacccggtggatatcaccgtggtggggccttccgccccgaccgtccaggccacttcggcggcgaacgagctggggacgaaggcgtcgtacaggaaccggggagcgtaggtgtggctgaccagctccaggcggccccggtcgctgagcacccggtactcctcgtcgaggctcatggtggcgcccacggcgctgccgatggcgacgccggtacggtgccggggcagccggtccttgtccaggccgctgtcgtcgaacgcctcccgggcggcgatcacggcgaactgcgcggcgcggtccatgcgccggatctcctgcgggccgaggccgagcgcctcggcgtcgaagtcgatctccgcggcgatgcgggagcggaagggggtcgggtcgaaggtgctgacggcgcgggtcgcggtgcggccggcggtgagcagttcccagaacgccttcgttccgactccgccgggggcgagcaccccgataccggtgacggcgacccgccgtccgccggcgccggggtgtgcgcggtccggtcgtacggtcatcgggacgcctgccaggaatagaagcgccgggccatcgcgtcggccggggagcgccaggtctcggggtcgtacgcgtcgatgaagggccgcaggtcggcgctgatccgctcgaagcgggggtcggtcttcgcctcctcgatccgctggtcgccgtcggccgcttcgaagtcctggaggtggaagtacagcccctggtaggagaagagctgccgccggcgggtgcccatccggtgcggcatctccgtggtgtcgaagtcccggaagagccggccgacgtcaccggccgacgaggggtccatccgggcgacgatcaacgtgctgtgcatggtggcttccttctttcgccggtctttcactggtcttccgtcggtcggtcagcgggccggaccgaaccagcggtccagggcctcggtgagtccggcgcggctgccgggcgagatccaggccacgtatccgtcgggcctgaccagcacggcccgggcgtcggacaacgggccctgcggtggggcgtcgtggagggaggcggtgacgatgtccacccggtcgctccagccagtggccgcctcgcgcacttccgcgtcgtcggcgatgtcgagcagaacgccccgggccgggtgcagcagttcggtggtgctggtctttccgtgggcccggaccagttcctggtggggcatgcgcatgccgagcaacgggtggtccccgccgtcgacttcgtagcggatgtccaggccggagaccatgccggccaggtgccgggagacctcgtcgtaccggatcagttcgctcagcacgtcccgcagcggctgcatctcgtcgccgctgaggaagagcatcccctgggcctgcgtgttcatcagcagcctgcgtcccaccgggtggcgctcctcgtggtacgtgtcgaggaggccggccggcgcacggcccgagaccacggccgcgagcttccagccgaggttcaccgagtcctgcacgctgacgttcatcccctggccccctgccgggaggtgcacgtgcgcggagtcgcccgcgagcagcacccggccgcgccggtaggcggacacctggcgggccggatccccgaaggcgctgacccagaccggttctccgtgcgagatgtcctgtccggtgagccgctgccacgcggcggcgacctcctggtacgggggcggcccggtgcggcggcgggcgggggcgccgcgctcgcagacgatgatgcggtcgaccccgtcgcccagcggcgcggacatcaccatcccgagcggcaccgtctcgccgatggggcgaggggtgatctcgcagccgcggatgtcggcgaggaacatctcccggctggcggaggttccggggaagtcgaacccggccgccttgcggaccgtgctgcgcccgccgtcgcagccgacgacgtagcgggtggtgaggctgcggggcccgtcggggccctcgacctccacgacgacgtgatcgccctcgtccgtgagagcccggaccgtatgaccccgcaggagttccgctccccgtccgagcgcccactcctccagtaccgattcggtcgtggactgcggaacggctttgacgccgtagtgcgctccttcgagtaccccgaagtcgaccggccggccgccgaagtgcccctgagtacttgtctcgacggggccgaaagcgggcagaattccgcgctggtcgaagacctccatggtccgggcggtgaaacccagaccgcgggactccccggtgcgttggggaagctgttcgagcaccatgacgtcgacgccgccgagccgcagctcaccggccagcatcagaccggccggacccgcgcccacgacaatgacagatgcatccatgtgtatctccctgggacagaaccgcggagcccggcgcataggccgtagaggctctcctgggatgtgctctgctggagcgaacgcttcgacagcagtggggaagttactccagagcttcttcgcaagcccgaagaaagtcaagcagcaatttcgcttgtccgggcccgcagcttcgcgcggaatcagggattaccggcttcgaagacagaactgaggaagctatccatgactctcttcggcgcggcctacagatatcaacaccgtcagtcgatggatatctcaagaaccgtcaggagggcgtgaagcgctgcatggacacgcaagaggaacgtcagagaaccgcaaagaagtgacctgcaccacacgcgggcggatgactccaacggatctgacgtggatcccacacttgtctgtgaatcatctgacgcagataaattcccgtgccgtcaccctcagggctggtcaagatgcattgcggccacgaaccgcccgtcgttagcctggccgcacgcatcgaaggagtgagcagatttgcccgagaagacgcccgtcgcggccacccgaacgaattgcccgggatacgcatgcccgacggcaccggtatccgagaaggcgccgggagccgggatcacagatatgcggagcgcccgggagacggccccgctgacgctgtgccgcgcgtgcatcgatttactgagggcgaccctgtgcgccctgccccagctgtacggggaatgcggccggctcctcaccggtgtcgtctctcctcggacggaaaggacgagcgggggtggccgggcgccgggaattccgctcaacacctcggctgtggaggcgcgttcggccatgatcgccacactcgcgtcgtgggccggcctggcggcggagtccggcgggcggcccggtccggaacggacggtgccggccctggcccgctggctcggcgaggaactgccccggatcgccgcgcacccggcggcgggcgagttctcgaaggaagtgcaccggctcgcggcgggggcccgccgggtggtgtcaccgggtccggcgcaccgggccaccgtcggcacctgcgtggagcccggctgcgacggaaagctcgtggccacgacgggggcgggcccgggcgggctgggcgagatccggtgcgacagggacggggcccactcctggacggagtacgactggagccggctgcgtcggcggctggccgcccggagcgcggtccgggcgggggctgccgcgggggccccggcgacccggtggctcgcgccccaggacgtctccctgctgtggcgcgtacccctcggcagtgtgtaccggctcgccagcgaacagagctggcggcgtgagcggcgcggggggcggtcctattacgacgagcaggacgtgcggcgcaccctcgacgggcgcctgaccgggcccgcgccctcctgacccggggaggcgtggccgccgccacctccaggcagcgcgcaggcgcgcccgcgacgggcgcgcccacgtacgcggtgccggcgggtcggcgtcaccggcaccgcgagcgttcagccgccgaagcggaaccccacgccccgtaccgtgatgatccactcgctggagccgagcttccccctgatcgaactggcgtgcgtgtcgatcgtgcgggtgagcccgccggcgaggcttccgctgcccggcttcaccccccagatctgccgcatcaactgctcacgggtgaagaccgtggccggctgggacgccagcagatgaagcagatcgaattccttgcgggtcagcttcacgggttcgtcgtccagatacacctgacgcccctcgccgtcgatacacaaggtcccgaaggacatcacacgatcgcggcgaacgctcgggctcgcgcgtcgcattaccgcgttgattcgggcgattaattcacggagaccgtacggcttcacaatacagtcgtcgcttcccgcctgcag

SEQ ID NO: 2 - protein, 411 aa, Streptomyces sp. H021
  “translate of OrfA, putative function ketosynthase I”
MLAPGGVGTKAFWELLTAGRTATRAVSTFDPTPFRSRIAAEIDFDAEALGLGPQEIRRMDRAAQFAVIAAREAFDDSGLDKDRLPRHRTGVAIGSAVGATMSLDEEYRVLSDRGRLELVSHTYAPRFLYDAFVPSSFAAEVAWTVGAEGPTTVISTGCTSGIDSVGYATELIREGSADIMIAGATDAPISPITLACFDAIKATTPRNEDPAHASRPFDATRNGFVLGEGSAVFVLEELGSALDRGAHVYAEISGYATRSNAYHMTGLRPDGTEMAEAIRVALDEARTDPADIDYISAHGSGTRQNDRHETAAFKKSLGEHARQVPISSIKSMVGHSLGAIGSIEIAACALAMEYDVVPPTANLHHPDPECDLDYVPLTARDWSTDSVLTVGSGFGGFQSAMVLTRPEGGAV

SEQ ID NO: 3 - protein, 409 aa, Streptomyces sp. H021
  “translate of OrfB, putative function ketosynthase II”
MSGTVVVSGLSVASPNGLGTQDYWSATTAGRSGIGPVSRFDASPYPAGLAGEVPGFVAEDHLPGRLLPQTDRMTRLALVVADWALADAGVRTEETPAYDMGVATASSSGGFEFGQNELRALWSKGSQYVSAYQSFAWFYAVNTGQISIRNGMKGPGSVVVSDQAGGLDAVAQARRQVRKGIPMVLTGGIDASLCPWGWVAQMSAGRLSTTRDPARAYLPFDAAAHGHVPGEGGALLVVEDEEAAHRRGARSLGEIAGYGSTFDPAPDSGRPPTLRTAIEIALRDAGITADDVDAVFADGAGLPDLDRAEALALTEVFGAGGVPVTVPKTMTGRLHSGAAPLDLACALLAMRDGVIPPTVNVAPSPGLALDLVCETRPARLATVLVLARGHHGFNSAMVLRAPRTRQGDR

SEQ ID NO: 4 - protein, 87 aa, Streptomyces sp. H021
  “translate of OrfC, putative function acyl carrier protein”
MTQRHFTFDDLRIILREAAGLEDDVLGDDALDAAFEDLGLESLALLETGSRIEREYGITLDDSSLTGSKTPRALLEVVNGELATAAA

SEQ ID NO: 5 - protein, 260 aa, Streptomyces sp. H021
  “translate of OrfD, putative function ketoreductase”
MPDQQRVALVTGGTSGIGLAVARLLGAQGHAVFIGARSADNVASTVKELRAEGLEADGLACDVRSGPEVAAFVAAAVDRYGTIDVLVNNAGRSGGGVTADIADELWDDVIATNLTSVFTVTREVLRAGGMREKSRGRIINIASTAGKQGVVLGAPYSASKHGVVGFTKALGNELAPTGITVNAVCPGYVETPMAQRVRQGYAAAYETTEEAVGAKFRAKIPLGRYSTPEEVAGLVGYLASDTAASLTSQALNVCGGLGNF

SEQ ID NO: 6 - protein, 489 aa, Streptomyces sp. H021
  “translate of OrfE, putative function oxygenase II”
MDASVIVVGAGPAGLMLAGELRLGGVDVMVLEQLPQRTGESRGLGFTARTMEVFDQRGILPAFGPVETSTQGHFGGRPVDFGVLEGAHYGVKAVPQSTTESVLEEWALGRGAELLRGHTVRALTDEGDHVVVEVEGPDGPRSLTTRYVVGCDGGRSTVRKAAGFDFPGTSASREMFLADIRGCEITPRPIGETVPLGMVMSAPLGDGVDRIIVCERGAPARRRTGPPPYQEVAAAWQRLTGQDISHGEPVWVSAFGDPARQVSAYRRGRVLLAGDSAHVHLPAGGQGMNVSVQDSVNLGWKLAAVVSGRAPAGLLDTYHEERHPVGRRLLMNTQAQGMLFLSGDEMQPLRDVLSELIRYDEVSRHLAGMVSGLDIRYEVDGGDHPLLGMRMPHQELVRAHGKTSTTELLHPARGVLLDIADDAEVREAATGWSDRVDIVTASLHDAPPQGPLSDARAVLVRPDGYVAWISPGSRAGLTEALDRWFGPAR

SEQ ID NO: 7 - protein, 109 aa, Streptomyces sp. H021
  “translate of OrfF, putative function cyclase”
MHSTLIVARMDPSSAGDVGRLFRDFDTTEMPHRMGTRRRQLFSYQGLYFHLQDFEAADGDQRIEEAKTDPRFERISADLRPFIDAYDPETWRSPADAMARRFYSWQASR

SEQ ID NO: 8 - protein, 315 aa, Streptomyces sp. H021
  “translate of OrfL, putative function cyclase”
MPAPGAHEVEHDITVSAPASAVYRLIAEVENWPRIFPPTIHVDRVEHDGARERIRIWATANGEAKHWTSRRTLDPEGLRIDFRQEVSTPPVAAMGGAWVVEPLGPDRSRVRLLHDYRAVDDDPEGLRWIEEAVDRNSRSELAALKDNVENAHAAEAWTFSFSDTVRATGSAEDMYDFIDEAQLWHERLPHVASVQLSQQPSGLQVLKMETRAKDGSTHLTESYRVCLPSERIAYKQTTLPALMSLHTGLWTFEQDGPEVVATSQHTVVLRTENIAAVLGPDAGVEEAREYVRSALSTNSRATLGHASDYARAQRR

SEQ ID NO: 9 - protein, 513 aa, Streptomyces sp. H021
  “translate of OrfM, putative function oxygenase I”
MVSAPDIDTQVLVVGAGPVGLMLAGEIALGGADVVVLERRTGPLTESRASTLHARTMELLDQRGLLEELGTPPAGGPGHFGGTRLDLTTDSPYSGQWKVPQVRTEELLEGWATRLGARVVKGHRLTDLRIAPHTVEATAEAEPGPGGAASALRVRAAYVVGCDGESSTVRELAGFAFPGRDATRELLRADVADVEVPDRRFERHPGGLAISARGPGGVTRIMVHEYGAPAGRDPGPPSFARVADTWRRVTGDDIGGGAPLWLNSFGDALRQASQYRRGRILLAGDAAHHQMPVGGQALNLGLQDAANLGWKLAAEVRGTAGPGLLDSYHAERHPAGRRVLDNIAAQALLLLGGSGTEQLRSLFGELLRKPRVRAHLAGTVAGVDVRYAAEPGAHRLVGARVPYLRFDTGIDTGIDTGIDTDTAAGPGSSFAALRAGRGVLVDLSGRPTPARAAVAPWQDGRVDVVSATPEPGCTAPEFTTALLRPDGHVAWADGTVPELEAALRRWFGPAARR

SEQ ID NO: 10 - protein, 253 aa, Streptomyces sp. H021
  “translate of OrfV, putative function reductase I”
MGKLANKTALVTGSSRGIGRATALRLAHEGALVAVHYATGEDAALSTVADIEAAGGRAFAVRSEFGVPGDIDTLFEALESGLKERTGVATLDILVNNAAIMGGVPPEEVTPELFDRLFAINAKAPFFIVQRALGLLADGGRIINISSGLTRFANPEEAAYAMTKGAVEQIALHFAKYLGARGITVNSVAPGITDNGSPVFDIPQAVEQMAQMSAFGQVGKPADVGDVVAFLASDDARWITGSFIDATGGTLLG

SEQ ID NO: 11 - protein, 194 aa, Streptomyces sp. H021
  “translate of OrfO, putative function reductase II”
MSQQPLHIAVLIGSVRTERFGHTVAAWFAELAAQRPDLTLDVIDLAELPFDLAGPSRTPTADMTALLARSRPRFQRADAYVVVTPEYNHSFPAALKNVIDWHLTEWAAKPVGFVSYGGIAGGLRAVEQLRQVFAELHAVTVRDTVSFHGGAATFDGPHPRDPESCGAAAKVLLDQLGWWGRALRQARADQPYGE

SEQ ID NO: 12 - protein, 332 aa, Streptomyces sp. H021
  “translate of OrfH, putative function dTDP-glucose-4,6-dehydratase”
MRMLVTGGAGFIGSRFVRMLLENRFEGWEDAEVVVLDALTYAGNRANLPASHPRLRFVRGDVCDTETVRELADGVHAVVHFAAESHVDRSLTGPAEFYRTNVLGTQSVLEAALRADVERVVHVSTDEVYGSISEGSWTEDRPLAPNTPYAASKAASDLVARSYWRTHGLDVSVTRCSNNYGPHQHPEKLIPLFATRLLRGMPVPLYGDGGNMREWLHVDDHCRALHLVLNKGRSGETYNVGGGNEWTNLDLTGRLLDLCGADESLVRRVADRKGHDLRYSLDDTKIREELGYAPQITFDQGLAETVAWYREHTDWWQDAAAVAERPDLSVLT

SEQ ID NO: 13 - protein, 436 aa, Streptomyces sp. H021
  “translate of OrfQ, putative function NDP-hexose-3-dehydratase”
MDPLDRKALLLDEVRKYHHEAQPDRRFVPGVTEIWPSGAVLDEDDRAALAEAALDMRIAAGVSSRRFESAFARRMGRRKAHLTNSGSSANLLSVSALTSHLLEDRRLRPGDEVITVAAGFPTTVNPILQNGLIPVFVDVEIGTYNTTADRVARAIGPRTRAIIIAHALGNPFEATEIAQLAADHDLFLIEDNCDAVGSLYDGQLTGTFGDLSTVSFYPAHHLTMGEGGCVLTANLALARIVESLRDWGRDCWCEPGESGKCLKRFEYQMGTLPVGYDHKYIFSHVGYNLKATDLQAALGLSQLAKLDEFCAARRRNWRRLRDGLADVPHLVLPEATPRSDPSWFGFVLTIDPEAPFSRAETVDFLESRKIGTRRLFAGNLTRHPAYIGQPQRVVGELTNSDLITDQTFWIGVYPALTDEMIDYVVASIREFVQARG

SEQ ID NO: 14 - protein, 187 aa, Streptomyces sp. H021
  “translate of OrfS, putative function NDP-hexose-2,3-dehydratase”
MPALSATTLTHPVLRPRREAATVERYARSAEALEGAAIRTADVPAWLEERRRAHDFRVDRIPFAELRGWDFAPDTGNLVHSSGRFFSVEGLDATVTDDDGTVTTWRQPIIKQPEVGILGLLVKEFDGVPHFLMQAKMEPGNPNLLQLSPTVQATRSNYTGAHRGAPVRYIDHFVSPEAGSVVADVLQ

SEQ ID NO: 15 - protein, 251 aa, Streptomyces sp. H021
  “translate of OrfR, putative function 4-ketoreductase”
MDIVGNGFLARHLRSLAGRHGGTLVLAAGVSWAAHTSPADFAREAALVEEKIAACLASGDRLVFFSTASTGMYGKVDGPGREDRPVVPCTPYGTHKLALEKRLEASGVDYLALRLGHLVGPGQPPHQLLPTLVRQMRTGTVHVHRGATRDLIDIDDVVTIVDRLLGAGLSREKVNVASGNAVPIERIIDHIEERTGLAARRVYRDRGGHHTISTDKLRALVPETEAMGFGPSYHRRVLDAFLTAAVPTATP

SEQ ID NO: 16 - protein, 397 aa, Streptomyces sp. H021
  “translate of OrfY, putative function O-acyltransferase”
MAPSNATSRLPSLTGMRFLAALLVFSVHGAAAGVFKDQGVAADYYRWFGNAGAVGVSFFFMLSGFVLTWSVRPADTVRGFWRRRLLKIFPNHLVTFVVAIVLLTVTSTAVAFPETLANLFLVHAWVPDSGYVETANTVSWSLSVELLFYLSFPLLIKGVSRIAPAVLWYAAGGVVLVIMLMPLIAQVLLPDTPGFMFMKISWTQIWFVYVFPVARLLEFVLGMLLARIVLSGRWIGLGVIPPALLTIAAYVGAVLIDHNPLYNYVAITVVPLALLIPAAAASDAKGRESLLSKRPMVWLGEISFAFYCVHYLILIYGHRLFGSDPNIFGKPSGPAWSTPGGLLFLAAALVVSVLAAWALYAIVERPVMRRWGRPPGPRRTEQTAVRPERAAEAPAAH

SEQ ID NO: 17 - protein, 137 aa, Streptomyces sp. H021
  “translate of OrfR1, putative function regulation
LQAGSDDCIVKPYGLRELIARINAVMRRASPSVRRDRVMSFGTLCIDGEGRQVYLDDEPVKLTRKEFDLLHLLASQPATVFTREQLMRQIWGVKPGSGSLAGGLTRTIDTHASSIRGKLGSSEWIITVRGVGFRFGG

SEQ ID NO: 18 - protein, 423 aa, Streptomyces sp. H021
  ”translate of OrfJ, putative function transporter“
MSRPPVPSKTGPSPGAPPRPWAARFLLLVLAGNMLIDGLEVSVVVVALPSIGADLGLSPWQVQWAMGGFALGFGALLLPGRLLVARFGHRRLYLGALALFVLASMAGGLTDDPLLLLGSRIVKGMCAALTAPTGLAIISTTYGEASARARALSVYTFCGGIGFTTGLLLAGALAPHDWHWTFVATAPVAAVLLVLAAAAVPRQQPGSAQPIAPTVRRLLGNGALLRPALGAAILNGTYLGLLSLMAHQAWDLLHWSPWQTAAAFLPACLPLAVTALSAGRVVARFGAPRLVVLGAALHLVGLLLYARLDLPRSYATDLLPTLALVGLGFVPAFAALNAQAGRAVGTADRGTATATYQTAVQAGAVAVPAAVAALLTFGPGDGGPVGDAAHRPVLLLLCALAALGLLVALRGLTARRPAVADDR

SEQ ID NO: 19 - protein, 266 aa, Streptomyces sp. H021
  ”translate of Orf1, putative function unknown
MRSARETAPLTLCRACIDLLRATLCALPQLYGECGRLLTGVVSPRTERTSGGGRAPGIPLNTSAVEARSAMIATLASWAGLAAESGGRPGPERTVPALARWLGEELPRIAAHPAAGEFSKEVHRLAAGARRVVSPGPAHRATVGTCVEPGCDGKLVATTGAGPGGLGEIRCDRDGAHSWTEYDWSRLRRRLAARSAVRAGAAAGAPATRWLAPQDVSLLWRVPLGSVYRLASEQSWRRERRGGRSYYDEQDVRRTLDGRLTGPAPS

SEQ ID NO: 20 - protein, 351 aa, Streptomyces sp. H021
  “translate of Orf2, putative function oxidoreductase”
VSMKYDLLGRTGVRVSELCLGAGTFGVEGWGASKEDALLMVDRYAQAGGNFIDTANVYGGGRSEECLGEVLAGRRDEFVLATKYNTMTRAGDVNSAGNHRKNLVASLENSLRRLRTDRVDVLWLHARDAFTPVEEVMRALDDQIRAGKVLYVGASNWPAWEVSRANMLAELRGWSAFAGLQVRYNLLERTAERDLLPMASACDVSAFAWGPLAEGRLTGKYLRGESGRLTVENWAGDDEHDTTVVREVVAVAEEGGWTPAQVALAWLRARPEAVLPILGATRPGQLQDSLAALDVRLDEEQTRRLDSLSEVSLGFPRELMRHGKFTQGAYSDRWPDLERRRGPGRTVATLL

SEQ ID NO: 21 - DNA, 19 bp, Artificial Sequence
  Description of Artificial Sequence oligonucleotide primer
tsgcstgcttcgaygsatc

SEQ ID NO: 22 - DNA, 20 bp, Artificial Sequence
  Description of Artificial Sequence oligonucleotide primer
tggaanccgccgaabccgct